(19) United States
(12) Patent Application Publication
(10) Pub. No.: US 2003/0008314 A1
(43) Pub. Date: Jan. 9, 2003

(54) Fiber-optic sensor array

(76) Inventors: Eric M. Priuska, Ann Arbor, MI (US); Richard H. Smith, Ann Arbor, MI (US); Judith L. Erb, Ann Arbor, MI (US); James G. Downward, Ann Arbor, MI (US); Otho E. Ulrich, Ann Arbor, MI (US)

Correspondence Address: KOHN & ASSOCIATES, Suite 410, 30500 Northwestern Highway, Farmington Hills, MI, 48334, US

(21) Appl. No.: 10/186,151

(22) Filed: Jun. 28, 2002

Related U.S. Application Data
(60) Provisional application No. 60/301,740, filed on Jun. 28, 2001.

(51) Int. Cl. C12Q 1/68; G01N 33/53; G01N 33/542

(52) U.S. Cl. 435/6; 435/7.9

(57) ABSTRACT

A method for performing a rapid, homogenous assays for monitoring the reactions of a binding target, by immobilizing a fluorescent-capable chelate complex that is derivatized so as to posses recognition binding ligands, labeling the complex with a labeled second chelator that is added to the assay thereby forming a fluorescent mixed chelate, and measuring the fluorescent mixed chelate, whereby the measurement of the label enable monitoring of the reaction of the binding target. A rapid assay for performing the above method including a first chelating molecule, a fluorescent-capable ion complexed with the first chelating molecule, a second chelating molecule for reacting with the fluorescent-capable ion complexed with the first chelating molecule, and a measuring device for measuring fluorescent resulting from the second is chelating molecule reacting with the fluorescent-capable ion complexed with the first chelating molecule. A biosensor for monitoring molecular interactions between receptors, including a biosensor having attached thereto a fluorescent-capable ion complexed with a first chelating molecule, whereby upon exposure to a second chelating molecule said complex becomes fluorescent is also provided.

### CROSS-REFERENCE TO RELATED APPLICATIONS

This application claims the benefit of priority under 35 U.S.C. Section 119(e) of U.S. Provisional Patent Application No. 60/301,740, filed Jun. 28, 2001, which is incorporated herein by reference.

### BACKGROUND OF THE INVENTION

1. FIELD OF THE INVENTION

The present invention relates to biological compound sensing technology. More specifically, the present invention relates to fiber-optic time-gated fluorometer and fiber-optic regenerable sensors embodying sensing technology and the extension of the technology to high-throughput screening using microwell assay techniques.

2. DESCRIPTION OF THE RELATED ART

In general, diagnostic tools used for detecting or quantitating biological analytes rely on ligand-specific binding between a ligand and a receptor. Ligand/receptor binding pairs used commonly in diagnostics include antigen-antibody, hormone-receptor, drug-receptor, cell surface antigen-lectin, biotin-avidin, substrate/enzyme, protein/protein and complementary nucleic acid strands. The analyte to be detected can be either member of the binding pair; alternatively, the analyte can be a ligand analog that competes with the ligand for binding to the complement receptor.

Numerous methods for detecting and/or quantifying ligand/receptor interactions have been developed. The simplest of these is a solid-phase format (e.g., radioimmunoassay) employing a reporter-labeled ligand whose binding to or release from a solid surface is triggered by the presence of analyte ligand or receptor. In a typical solid-phase sandwich type assay (e.g., enzyme-linked immunosorbent assay or ELISA), the analyte to be measured is a ligand with two or more binding sites, allowing ligand binding both to a receptor, e.g., antibody, carried on a solid surface, and to a reporter-labeled second receptor. The presence of analyte is detected (or quantified) by the presence (or amount) of reporter bound to the solid surface. In a typical solid-phase competitive binding assay, an analyte ligand (or receptor) competes with a reporter-labeled analyte analog for binding to a receptor (or ligand) carried on a solid support. The amount of reporter signal associated with the solid support is inversely proportional to the amount of sample analyte to be detected or determined. Unfortunately, such conventional methods suffer from numerous disadvantages in that they cannot be read in real time because a wash step is required to remove reporter-labeled ligand from the solid surface prior to reading the reporter signal. If this is not done, reporter bound to the solid surface cannot be distinguished from reported in the surrounding solution. This results in a requirement for long incubations in order that equilibrium is approached and the value of the reported signal not change appreciably altered during the time elapsed during the wash steps. This makes true kinetic observation of molecular interactions impossible and increases the time required before assay results can be reported.

Quantitative binding assays of this type involve three separate components: a reaction substrate, e.g., a solid-phase test strip, a solution containing the reporter-labeled ligand and a separate reader or detector device, such as a scintillation counter or spectrophotometer. The substrate is generally unsuited to multiple assays, or to miniaturization, for handling multiple analyte assays from a small amount of body-fluid sample.

Recently, a variety of electrochemical biosensors have been developed for facile, point-of-care detection and/or quantification of ligand receptor binding events. Generally, a biosensor is composed of (i) a biochemical receptor, which uses receptors such as enzymes, antibodies or microbes to detect an analyte, and (ii) a transducer, which transforms changes in physical or chemical value accompanying the reaction or binding event into a measurable response, most often an electrical signal. Several biosensors based on immobilized enzymes are available commercially and are especially useful in clinical analysis. The term immunosensor is used when an antibody or antigen is immobilized to interact respectively with its specific binding partner (i.e., a target antigen or a target antibody).

The conversion of the biological recognition (binding) event to a quantitative result has been accomplished by a variety of techniques, including electrochemical, calorimetric, and optical detection. Two basic approaches are common. In the first, ligand binding assays uses chemical labels, such as radioisotopes, fluorescence, or reporter enzymes. The use of such labels can alter the properties of the labeled species. A second approach is seen in mass based optical sensors. These require no labeling of the binding molecules since the signal which is transduced is the change in a mass-related variable, such as refractive index, resulting from binding of molecules to a partner affixed to the surface of the sensor. While this nicely avoids the problems associated with labeling the binding molecules with a reporter, the accuracy of these methods is compromised by their sensitivity to nonspecific interactions between other components in the sample and the binding surface.

By developing an assay that uses an immobilized fluorescent chelate complex that is derivatized and possesses recognition binding ligands, both weaknesses are avoided. Since the fluorescent label resides on the surface rather than on the binding molecule, the integrity of biological response is not compromised by coupling to a reporter. Because the fluorescence of the surface is not affected by random binding to the surface and transduction is not based upon mass, the accuracy of the assay is not so easily compromised. The recognition binding ligand can be identical to the target analyte in a simple competitive assay. In drug discovery, the ligand can be a lead compound already known to interact directly with a receptor or other binding species of interest.

Fluorescent and luminescent chelates have been previously used as reporter moieties which are coupled to one of the binding partners. They offer high sensitivity and have been commercialized by Wallac Oy (Turku Fl) as “Delphia. ” The shortcomings of this instrument include those already mentioned and additionally fluorescence is only revealed by extraction of the europium complex into a micelle where there is sufficient hydrophobicity. The problem of extraction has been circumvented by development of compounds such as the europium chelator 4,7- bis(chlorosulfophenyl)-1,10-phenanthroline-2,9-dicarboxylic acid (BCPDA) which is fluorescent in aqueous solution. The molecule is adapted to be easily coupled to proteins for use as a reporter. A portion of the metal/chelate complex described in the current invention resembles BCPDA. The uniqueness of this invention is that the chelate complex is mixed rather than homogenious, being only partially comprised of BCPDA-type molecules and partially comprised of nonfluorescent chelator affixed to the solid phase. In addition the mixed chelate complex is not attached as a reporter label to a binding molecule in solution, but rather is permanently in place on the solid surface in a manner which causes binding to a partner molecule to modulate the fluorescent signal already present. Not only does this avoid problem of loss of biological activity of the molecules in the solution phase, but the presence of an initial fluorescent signal also provides a convenient reference point for normalization between samples.

### SUMMARY OF THE INVENTION

According to the present invention, there is provided a method for performing a rapid, homogenous assays for monitoring the reactions of a binding target, by immobilizing a fluorescent-capable metal ion chelate complex that is derivatized so as to posses recognition binding ligands, labeling the complex with a second chelator that is added to the assay thereby forming a fluorescent mixed chelate, and measuring the fluorescent mixed chelate, whereby the measurement of the label enables monitoring of the reaction of the binding target. Also provided is a rapid assay for performing the above method having a first chelating molecule, a fluorescent-capable ion complexed with the first chelating molecule, a second chelating molecule for reacting with the fluorescent-capable ion complexed with the first chelating molecule, and a measuring device for measuring fluorescence resulting from the second chelating molecule reacting with the fluorescent-capable ion complexed with the first chelating molecule. A biosensor for monitoring molecular interactions between receptors, the biosensor having a biosensor having attached thereto a fluorescent-capable ion complexed with a first chelating molecule, whereby upon exposure to a second chelating molecule said complex becomes fluorescent is also provided.

### DESCRIPTION OF THE DRAWINGS

Other advantages of the present invention are readily appreciated as the same becomes better understood by reference to the following detailed description when considered in connection with the accompanying drawings wherein:

FIG. 1 shows a time resolved UV biosensor fluorometer was used with a europium chelate;

FIG. 2 is a diagram showing the sensor fiber surface covered with chelators having attached EREs;

FIG. 3 is a graph showing the sensor fibers Eu chelate is saturated with Eu⁺³;

FIG. 4 is a graph showing the binding of biological receptor reduces fluorescence of immobilized ligand-DTPA-(Eu⁺³)-PDA complex;

FIGS. 5 A and B are graphs showing that in the absence of ligand, hER-β displays minimal binding to the vitellogenin ERE consensus sequence and hER-α displays rapid, unstable binding to the vitellogenin ERE consensus sequence, further, binding between hER-α and the ERE in the absence of estrogen has often been noted in the literature;

FIGS. 6 A and B are graphs showing that in the presence of anti-E1g antibody, fluorescent signal drops, due to the displaced (Eu⁺³)-PDA complex (FIG. 6A) and upon the addition of antibody plus 1 mM E1g (FIG. 6B), no more antibody binds and the bound antibody is released;

FIG. 7 is a diagram showing the mechanism of the present invention is fluorescent assay;

FIG. 8 is a graph showing that using the same type of E1g fiber, the addition of estrogen receptor causes a drop in fluorescent signal and upon the addition of receptor-free buffer, the signal rises again, yielding data for the on and off rates of the estrogen receptor to estrone glucuronide; and

FIGS. 9A and B are graphs showing that the estrogen receptor has a fast kinetic on rate for the ERE (estrogen response element), in the presence and absence of ligand, but has a much lower off-rate in the presence of the ligand DES.

### DESCRIPTION OF THE INVENTION

Generally, the present invention provides an assay and method for use in performing rapid, homogenous assays for monitoring the reactions of a binding target. The reactions can include, but are not limited to, the binding of a first compound to a second specific recognition binding target, or the influence of a third compound on the binding of the first compound to its second recognition binding target. The monitoring can occur in real time. The fiber-optic time-gated fluorometer described herein can be used with fiber-optic biosensors incorporating the assay technology described herein. This assay technology can also be used in a variety of other sensing formats for use with single samples or it can be used as a multiple sample screening assay to identify specific compound behaviors by using it with a microwell fluorescence plate reader.

By “chelating molecule,” as used throughout the application, it is meant any chelating molecule that can form a coordination complex with metal ions such as and without limitation, one that has multiple pendent carboxyl groups. One example of such a chelating molecule is DTPA (diethylenetriaminepentaacetic acid).

The chelating molecule, must have the following features:

1) It forms a coordination complex (or chelate ) with a metal ion; and

2) It possesses reactive moieties through which both of the following can be achieved:

a) covalent coupling of the molecules on a sensor surface to the chelating molecules; and

b) covalent coupling of the chelating molecules to molecules having binding affinity for a molecule of interest.

The simultaneous possession of all of these characteristics produces a situation in which chelation of a metal ion is likely to be disrupted by binding between molecules in solution having affinity for the molecules that are attached to the chelate and the molecules attached to the chelating molecules.

The “fluorescent-capable” ion is capable of forming fluorescent chelates, either alone or in conjunction with another chemical. For example, europium is representative of an ion that is a rare earth or lanthinide that is capable of forming fluorescent chelates, either alone or in conjunction with another chemical. The ion must be capable of being chelated by multiple chelating moieties such as and without limitation pendent carboxylic moieties or aromatic nitrogen compounds such as phenanthroline.

The fluorescent-capable ion, or other similar ion, must have the following features:

1) It can be chelated by the molecules attached to the fiber;

2) It is fluorescent or illuminescent under certain conditions of chelation; and

3) The fluorescence or illuminescence of the chelated metal is modulated by proximity of the chelate to a large molecular complex forming on the fiber surface. This can be caused either by the metal or the fluorescent chelator being forced out of the chelate by the larger complex, or by change in the hydrophobicity or pH of the micro-environment surrounding the fluorescent chelate, or perhaps by some other unknown mechanism. The important feature is that the complex modulates the fluorescence.

In the case of the DTPA-Eu complex, the complex is not fluorescent. PDA (4,7-diphenyl-1,10-phenanthroline-2,9-dicarboxylic acid) is introduced into the surrounding solution because PDA-Eu is fluorescent. Because Eu⁺³ has nine coordination sites, it was possible to form a mixed chelate with DTPA-Eu-PDA that was fluorescent. If molecules that meet the conditions for which the metal chelate is also fluorescent can be created, then the PDA can become unnecessary. The present invention applies in both circumstances: one in which the chelate complex is fluorescent and one in which it is not, but with which a fluorescent mixed chelate can subsequently be formed by addition of additional chelator molecules in the solution.

The term “PDA” as used herein is intending to include a compound having the following features, which are exemplified by PDA:

1) It forms fluorescent chelates with metal ions; and

2) It is water soluble.

The present invention provides a method for monitoring the reactions of a binding target. Typical ligand binding assays require the use of chemical labels, such as radioisotopes, fluorescence or reporter enzymes. The use of such labels can alter the properties of the labeled species. The method described herein utilizes an immobilized chelate complex that is derivatized so as to posses recognition binding ligands. The complex becomes fluorescent when a second chelator is added to the assay and forms a mixed chelate with the DTPA-Eu. The mixed chelate creates a detectable label that is measurable. The measurement of the label is conducted using methods known to those of skill in the art depending upon the label that is utilized. Preferably, the label is a fluorescent label that is measured using ultra-violet light (UV).

With regard to the immobilized chelate complex, preferably, a non-fluorescent compound is used. One example of such a compound is DTPA (diethylenetriaminepentaacetic acid) derivatized with the ligand E1-g (estrone-3-glucuronide) that is complexed with europium (Eu⁺³) and chemically immobilized to the surface of an optical sensor fiber. This complex becomes fluorescent when a second chelator, PDA (4,7-diphenyl-1,10-phenanthroline-2,9-dicarboxylic acid) present in the solution forms a mixed chelate with the Eu⁺³ on the fiber surface. Alternately, other ligands have been used, such as the estrogen-receptor response element (ERE) DNA consensus sequence that is derivatized to the DTPA-Eu⁺³-PDA fluorescent chelate complex at approximately the same location, so -as to provide a direct means of observing the effect that a wide variety of test compounds have on the binding of hER-α or hER-β receptors to the nuclear ERE.

The presence of the Eu⁺³ species, loosely bound within the mixed chelate complex, allows fluorescence to occur when the energy absorbed from UV excitation within the absorbance passband of the PDA chelating molecule (the antenna molecule) that is around 337 nm is transferred to a Eu⁺³ atom (the receiver) that fluoresces at a wavelength around 613 nm. Conversely, if Eu⁺³ is not present within the mixed chelate complex, fluorescence at 613 nm can not occur. Moreover, if the amount of Eu⁺³ bound to the chelate decreases, observed fluorescence decreases. For example, it is observed that after loading the chelate complex bound to a sensor fiber with Eu⁺³ such that maximum fluorescence is achieved, that sensor fluorescence can slowly, but steadily, decrease if buffer not containing Eu⁺³ is made to flow past the sensor fiber. Fluorescence is also experimentally observed to decrease as ligands within a solution bind to the ligand binding sites that have been derivatized to the chelate molecule, thus causing fluorescence from the sensor fiber to decrease.

To make the fluorescence measurements reported herein, a pulsed nitrogen laser and a time-resolved fluorometer were used (FIG. 1). It is also possible to stimulate chelate fluorescence occurring around 613 nm using other light sources capable of illuminating the chelate within the absorbance passband of Eu⁺³.

Since fluorescence is routinely observed to decrease when a first substance within a solution surrounding the sensor fiber binds to a variety of different ligands derivatized to the chelate complex bound to the fiber surface, the bound ligands are thought to have changed the steric conformation of the chelate fluorophore complex in some manner such that the europium becomes released from the chelate. The chelate-bound europium is released back into solution until a new equilibrium state is reached and overall fiber fluorescence is thereby diminished. While decreased fluorescence was observed where the first substance is an antibody or the estrogen receptor and the second substance is E1-g or the ERE, the choice of binding pairs (one in solution, one attached the Eu⁺³-chelate complex) that causes an observed decrease in fluorescence is quite broad and, as a result, the assay format described herein has broad application.

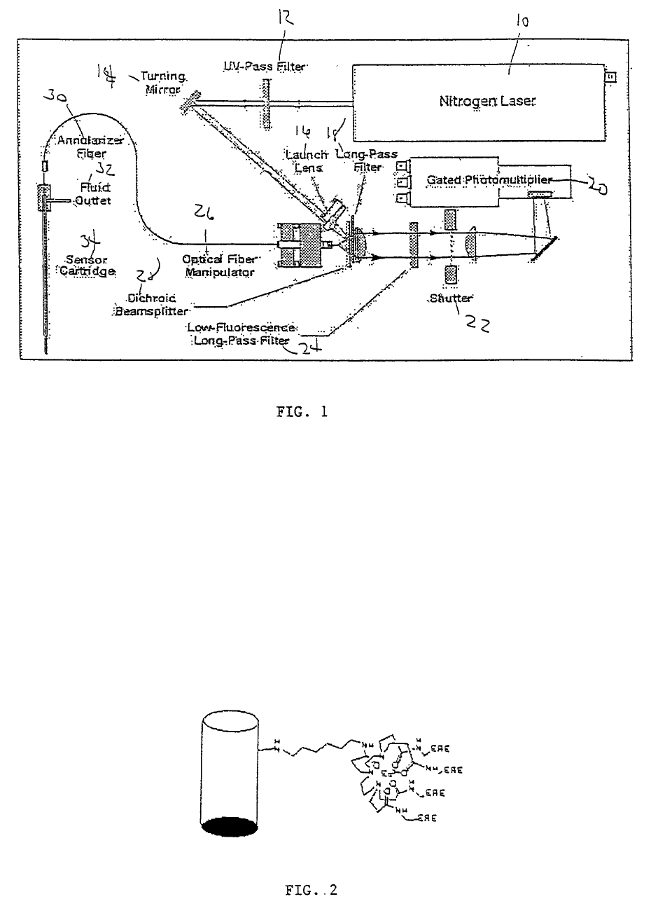

More specifically, an E1g-DTPA complex or an ERE-DTPA complex coupled to optical fibers forms a chelate with Eu⁺³ ions present in the solution surrounding the fiber. FIG. 2 shows a fiber surface with EREs attached to a surface-bound chelator. PDA is also present in the solution and can chelate with and acts as an antenna for Eu⁺³. However, because the fiber is part of an evanescent sensing apparatus that only “sees” fluorescence from surface-bound molecules, measured fluorescence is directly proportional to the number of PDA-(Eu+3)-DTPA mixed chelate complexes that form on the fiber surface.

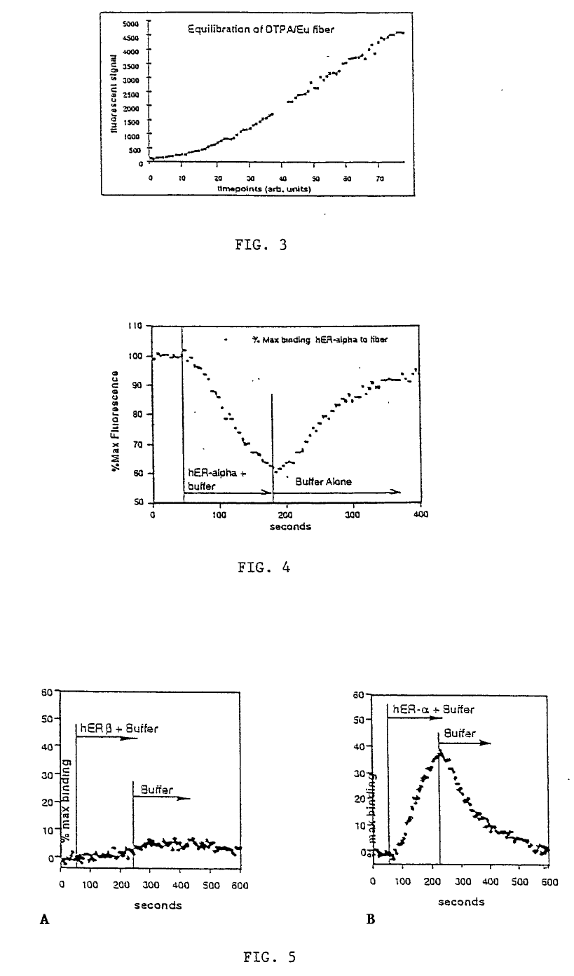

Prior to use, the sensoris surface bound chelators must be loaded with europium. In practice, the sensor fiber can be supplied pre-loaded with europium. However, for the experiments described herein, the fluorescence present on the fiber was monitored while a buffer solution containing PDA and Eu⁺³ was flowed through the sensor cartridge. At the point at which fluorescence ceased to increase, it is assumed that equilibrium had been reached and all available surface bound chelate molecules were loaded with Eu⁺³. If buffer not containing Eu⁺³ was flowed past the sensor, a decrease in observed fluorescence occurred over a similar time scale as governed by the dissociation rate for the Eu⁺³ chelate complex. Typical fluorescence measured while loading of a sensor fiber with Eu⁺³ is shown in FIG. 3.

Once loaded with Eu⁺³, initial sensor fiber fluorescence readings are made and sample is added to the PDA and Eu+3 buffer that contains specific binding species, such as antibodies directed to E1-g, or the estrogen receptor directed toward E1-g or the ERE. The binding events results in reduction of fluorescence, as previously discussed. Data illustrating this phenomenon are presented in FIG. 4.

The present invention also provides a regenerable, label-free, evanescent fiber-optic biosensor for monitoring molecular interactions between receptors and an assay incorporating the same. One example of such receptors are estrogen receptor modulators and both human estrogen receptor α (hER-α) and human estrogen receptor β (hER-β). The biosensor of the present invention is both highly specific and reusable, and requires only 10⁻¹⁴ moles of receptor.

The present invention can be extended to any binding assay involving a free binding species (antibody, receptor, imprinted polymer, aptamer, phase display peptide, etc.) and a derivative or analog of the primary analyte molecule attached to a specifically designed chelate fluorophore. Additional extensions and embodiments of this invention are presented in Examples 1 and 2. The biosensor uses an immobilized chelate complex that is derivatized so as to posses recognition binding ligands. For example, in the present invention, a fluorescent compound was used that is made of DTPA (diethylenetriaminepentaacetic acid) derivatized with the ligand E1-g (estrone-3-glucuronide) that is complexed with europium (Eu⁺³) and chemically immobilized to the surface of an optical sensor fiber. This complex becomes fluorescent when a second chelator, PDA (4,7-diphenyl-1,10-phenanthroline-2,9-dicarboxylic acid) present in the solution forms a mixed chelate with the Eu⁺³ on the fiber surface. Alternately, other ligands have been used such as the estrogen-receptor response element (ERE) DNA consensus sequence that is derivatized to the DTPA-Eu⁺³-PDA fluorescent chelate complex at approximately the same location, so as to provide a direct means of observing the effect that a wide variety of test compounds have on the binding of hER-α or hER-β receptors to the nuclear ERE.

The present invention also provides a fiber-optic, time-gated fluorometer apparatus that is used to measure fluorescence using the fiber-optic biosensors of the present invention.

The fiber-optic time-gated fluorometer of the present invention is shown in FIG. 1. This fluorometer is used with fiber-optic biosensors incorporating fluorophores, described more detail in Example 1, that are excited by ultra-violet (UV) light and that have a long fluorescence lifetime. Since these fluorophores require the excitation wavelength to be in the UV range, the fluorometer of the present invention uses a source of UV light such as, but not limited to, the nitrogen laser shown in FIG. 1 that operates at a wavelength of 337 nm. Similar to the fluorometer described in previous patents to Applicants (U.S. Pat. Nos. 5,854,863 and 5,952,035), the fluorometer of the present invention utilizes annularizing optics to provide strong evanescent coupling between the exciting laser light and the fluorescent label. Because UV radiation is used, completely different illumination and fluorescence measurement optical systems are required to achieve both high sensitivity and a high signal to noise ratio (SNR).

Because UV illumination is used to excite the fluorophores, the fluorometeris optics are designed to eliminate or minimize self-fluorescence caused by the UV laser pulse. This is done in several ways. First, UV transmissive optics are made from quartz or fused silica. Second, the UV beam is injected off-axis into the annularizer fiber in a manner such that it does not pass through the large numerical aperture focusing doublet (that can not be made out of fused silica) used to collect fluorescence from the sensor. Third, long wavelength pass band filters are used to minimize residual UV radiation or fluorescence below 600 from reaching the detection system.

Minimizing sources of fluorescence can not, by itself, provide the fluorometer with as high an SNR as is needed. The previously mentioned fiber-optic fluorometers (U.S. Pat. Nos. 5,854,863 and .5,952,035) achieve a high SNR by employing a holographic notch filter block the exciting near-IR laser radiation propagating back to the photodiode detector by a factor of>10⁶. Because UV holographic notch filters do not yet exist, the present invention employs long-lived fluorophore labels and time-gated detection means such as, but not limited to, time-gated photon counting, to prevent detection of fluorescence signal that can be produced from sources other than the fluorophore label.

For example, for the Europium chelate fluorophore described herein, five fluorescence peaks at 613 nm were detected and the fluorophore has a long-lived decay time of hundreds of microseconds whereas the lifetimes of optical component or organic compound fluorescence is at least 1000 times shorter. Thus, by measuring only fluorescence occurring long after any biological and optical fluorescence has decayed to zero, it is possible to measure, with high discrimination, only fluorescence from the Europium fluorophore label. This approach has the additional benefits that counting noise caused by the electrical discharge used to pulse the nitrogen laser and UV laser radiation reflected from the face of the annularizer, is also blocked.

To measure only long-lived fluorescence, the fluorometer shown in FIG. 1 uses time-gated photon counting. A control system employing a combination of a computer controller, software, and electronic timing apparatus is used to control the timing and gating of the laser and data acquisition system. Prior to triggering each laser pulse, control software disables the photon detectors, such as but not limited to, photomultipliers, and the counting electronics. To compensate for pulse-to-pulse intensity variation in light output, a beam splitter and a second photon detection device is used to count the number of UV photons delivered in each light pulse. This allows the biosensoris response vs. time to be normalized to a constant laser output value. At each laser pulse, delay electronics are triggered, which, after the delay time set by the control program, generates a pulse to gate on the photon detectors and counting electronics. After a specified time has elapsed, the control software acquires fluorescence measured using the counting electronics.

The present invention also provides an assay and method for measuring free ligand, and/or a method for measuring the concentration or activity of the receptor or other binding species. There is no need for labeling of the receptor, which can have altered binding characteristics due to the labeling event. In the case of the estrogen receptor, labeling methods that target primary amine functions can sharply reduce receptor activity, as the ligand binding site contains a primary amine. Various assay formats can result from the use of this method. In one method, various substances that can bind to the estrogen receptor are tested, using the format described above.

The present invention can also utilized as a simple, rapid and real-time screening method, which is amenable for high-throughput screening activities and can thereby allow compounds in a library to be rapidly screened for binding to a candidate target molecule or for a library of compounds to be checked for possible interactions between a specific biomolecule and its recognition target. The screening methods are easily adapted to mass screening using a 96-well or larger plate and a fluorescence plate reader using techniques known to those of skill in the art.

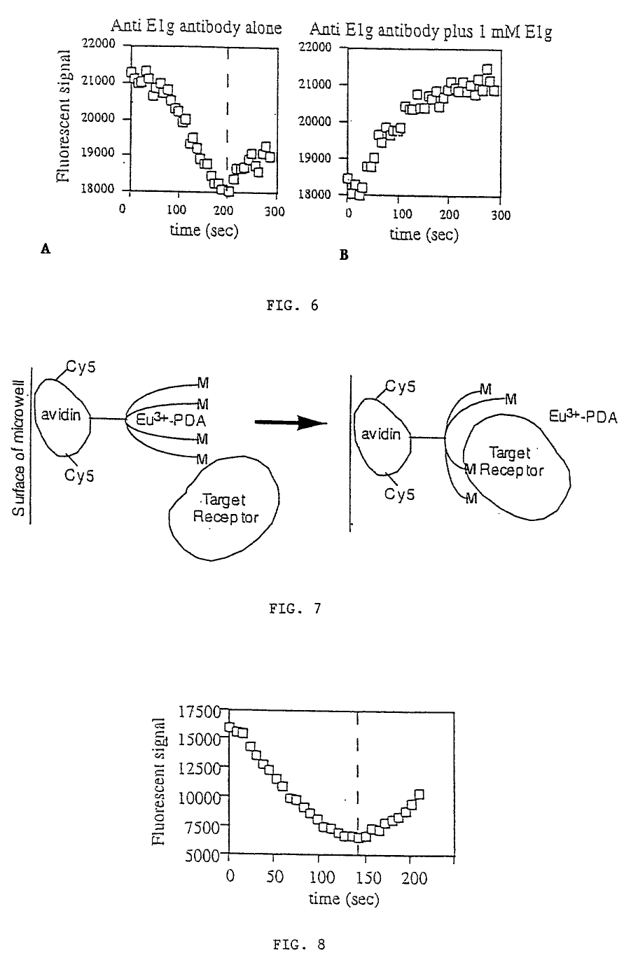

The present invention depends on the use of a ligand-derivatized chelator, diethylenetriaminepentaacetic acid (DTPA), or related species, which can be coupled to microtiter plates (or other surfaces) and loaded with europium (Eu⁺³) to form a chelate complex. In the preferred embodiment, to identify ligands for estrogen receptors, the ligand is E1g (estrone-3-glucuronide), although it is recognized that other ligands for estrogen receptors exist, and that other receptors, such as androgen receptors, can use other specific ligands. The resulting ligand-DTPA-(Eu⁺³) complex becomes fluorescent when the antenna molecule, PDA (4,7-diphenyl-1,10-phenanthroline-2,9-dicarboxylic acid), present in the solution, forms a mixed chelate ligand-DTFA-(Eu⁺³)-PDA complex and when this mixed chelate complex is illuminated with light within PDAis absorbance band. In the experiments, a nitrogen laser operating at 337 nm was used, but other wavelengths within the PDAis absorbance band can also be used. The binding of biological receptor to the immobilized ligand bound to the mixed chelate complex results in reducing the fluorescence of the complex, as illustrated in the FIGS. 6 and 8. Thus, by this means ligand-receptor binding can be measured without the need for chemical derivatization of the biological receptor. This combination of the immobilized fluorescent complex and the biological receptor provides the basis for the screening assay. A figure illustrating this principle when used on a fiber sensor surface is shown in FIG. 2.

The assay also requires coupling a known ligand to the chelator. The ligand must have a functional group available that permits chemical coupling without greatly diminishing binding strength for the receptor. Because many such ligand-receptor couplets have been identified, it seems reasonable to suggest that suitable ligands can be found that can allow the synthesis of binding complexes specific for virtually any known receptor. Alternative coupling chemistries can be adopted, such as the substitution of other chelators for DTPA, other lanthanides for europium, and other chelating antenna molecules for PDA. For this assay to provide the desired (sterically derived) signal, the ligand must become bound in such a way that the stereochemistry of the mixed chelate-(Eu⁺³) complex is disturbed so as to cause (Eu⁺³) to become unbound from the mixed chelate, or to cause the separation of the antenna molecule and the (Eu⁺³) to significantly change. Thus, it can be that certain ligand-receptor couplets, such as receptors for large peptides, can not respond to this assay.

In an envisioned embodiment of this invention, microtiter plates are prepared in which the ligand-DTPA-(Eu⁺³) chelate complex is immobilized to the surface of each well. At this point, only two addition steps are required to perform the assay. In the first, the sample compounds in a solution containing PDA and (Eu⁺³) are added to the various wells, each at different concentrations or dilutions, as desired. The assay is initiated in the second step by the addition of the biological receptor. To measure assay results, plates are placed in fluorescence plate readers capable of making time-resolved measurements. Since the specific fluorescent species is found only on the surface of each well, and not in solution, and since receptor binding to the complex specifically reduces fluorescence, fluorescence changes also can be measured in real. time, which can allow determination of kinetic binding constants.

Certain features of this method should be noted. For example, the inherent fluorescence of the ligand-DTPA-(Eu⁺³)-PDA mixed chelate complex provides a simple quality control measure, or normalizing value, allowing changes in each well to individually be calibrated. In addition, due to the real-time nature of the method, matrix effects that can be imparted by the sample on the ligand-DTPA-(Eu⁺³)-PDA complex can easily be monitored, simply by measuring fluorescence over time after sample addition.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations that become evident as a result of the teaching provided herein.

### EXAMPLES

### Example 1

This example demonstrate the use of the present invention to create a regenerable, label-free, evanescent fiber-optic biosensor for monitoring in real-time, molecular interactions between estrogen receptor modulators and both human estrogen receptor α (hER-α) and human estrogen receptor β (hER-β). This biosensor is both highly specific and reusable, and requires only 10-14 moles of receptor. This invention can be extended to any binding assay involving a free binding species (antibody, receptor, imprinted polymer, aptamer, phase display peptide, etc.) and a derivative or analog of the primary analyte molecule attached to a specifically designed chelate fluorophore. Additional extensions and embodiments of this invention are presented in Examples 2 and 3.

Method:

The present invention can be used as an assay method for free ligand, or as a method for measuring the concentration or activity of the receptor or other binding species. There is no need for labeling of the receptor, which can have altered binding characteristics due to the labeling event. Using estrogen receptor, labeling methods that target primary amine functions can sharply reduce receptor activity, as the ligand binding site contains a primary amine. Various assay formats can result from the use of this method. In one method, various substances that can bind to the estrogen receptor are tested, using the format described above. Estrogenic substances can quantitatively reduce the binding of receptor to the E1-g/DTPA, which can result in higher signal than can be measured in the absence of free ligand. In a second method, the assay format can be used as a method to screen for the presence of receptors, antibodies and other binding species. Sample solutions containing these species can reduce the fluorescence inherent in the immobilized E1-g/DTPA/Eu complex. Therefore, this method provides a simple means to screen for binding activity during fractionation and other experimental operations.

Results:

The use of a sensor fiber to measure the binding kinetics of hER-α and hER-β to ERE attached to the chelate bound to the sensor fiber surface is shown in FIG. 5.

### Example 2

This example demonstrates the use of the present invention as a rapid assay method that can allow performing rapid, homogenous assays for monitoring the binding (possibly in real-time) of a first compound to a second specific recognition binding target, or the influence of a third compound on the binding of the first compound to its second recognition binding target. The assay can be used with single samples, or it can be, used as a mass sample screening assay to identify specific compound behaviors by using it with a microwell fluorescence plate reader.

Method:

Various assay methods are used in screening libraries of compounds produced in the pharmaceutical discovery process for specific biomolecular behaviors. Since pharmaceutical agents are sought that typically bind selectively to specific biological moieties such as receptors, enzymes, DNA sequences, etc., while possessing minimal binding to other biological moieties, measurement of the binding event alone can provide a simple and rapid screening tool for candidate drugs. In addition, the potential of a candidate molecule to interfere with a variety of other biological systems should be known, preferably before clinical trials have begun. This example demonstrates a simple, rapid and real-time screening method, which is amenable for high-throughput screening activities and can thereby allow compounds in a library to be rapidly screened for binding to a candidate target molecule or for a library of compounds to be checked for possible interactions between a specific biomolecule and its recognition target. Both screening methods are easily adapted to mass screening using a 96-well or larger plate and a fluorescence plate reader.

Typically, ligand binding assays require the use of chemical labels, such as radioisotopes, fluorescence or reporter enzymes. The use of such labels can alter the properties of the labeled species. An immobilized fluorescent chelate complex is derivatized so as to posses recognition binding ligands. Such a ligand can be identical to the target analyte in a simple competitive assay, or in drug discovery, can be a lead compound already known to interact directly with a receptor or other binding species of interest. DTPA (diethylenetriaminepentaacetic acid) has previously been derivatized with E1g (estrone-3-glucuronide) that is complexed with europium (Eu⁺³) and chemically immobilized to the surface of an optical sensor fiber. This becomes fluorescent when a second chelator, PDA (4,7-diphenyl-1,10-phenanthroline-2,9-dicarboxylic acid) present in the solution forms a mixed chelate with the Eu⁺³ on the fiber surface. Alternately, applicants have used other ligands such as the estrogen-receptor response element (ERE) DNA consensus sequence that is bound covalently to the DTPA-(Eu⁺³) fluorescent chelate complex at approximately the same location, to provide a direct means of observing the effect that a wide variety of test compounds have on the binding of the human estrogen receptor alpha or beta (hER-α or hER-β) receptors to the nuclear ERE.

The presence of the Eu⁺³ species bound within the chelate complex allows fluorescence to occur when the energy absorbed from UV excitation within the absorbency passband of the organic chelating molecule (the antenna molecule), which for PDA is around 337 nm, is transferred to a Eu⁺³ atom (the acceptor) that fluoresces at a wavelength around 613 nm. Conversely, if Eu⁺³ is not present within the chelate complex, fluorescence at 613 nm can not occur. Moreover, if the amount of Eu⁺³ bound to the chelate decreases, observed fluorescence decreases.

To make the fluorescence measurements reported previously, a pulsed nitrogen laser and a time-resolved fluorometer were used (FIG. 1) to stimulate fluorescent chelate molecules bound to the surface of the fiber sensor (FIG. 2). FIG. 1 depicts a time resolved fluorometer that includes the following operative connected: a nitrogen laser 10, a UV pass filter 12, a turning mirror 13, a launch lens 16, the material then passes through a dichroic beamsplitter 28 and a long pass filter 18, then through a low-fluorescence long pass filter 24, and through a gated photomultiplier 20 via a shutter 22. The material then passes through an optical fiber manipulator 26 to an annularizer fiber 30, to a fluid outlet 32 and then to a sensor cartridge 34. Because an evanescent fiber-optic sensor was used, Eu⁺³ fluorescence was only seen if was produced by a chelate molecule attached to the sensor surface.

In the present invention, an E1g-DTPA complex or an ERE-DTPA complex coupled to optical fibers forms a chelate with Eu⁺³ ions present in the solution surrounding the fiber. FIG. 2 shows a fiber surface with EREs attached to a surface-bound chelator. PDA is also present in the solution and can chelate with and acts as an antenna for Eu⁺³. Because the fiber only “sees” fluorescence from surface-bound molecules, measured fluorescence is directly proportional to the number of PDA-(Eu⁺³)-DTPA mixed chelate complexes that form on the fiber surface.

To provide a demonstration of the effect of specific binding protein on the fluorescence emission of the an E1-g/DTPA/Eu/PDA complex, experiments were performed on optical fibers derivatized with the complex. A fiber (FIG. 2) was mounted in a flow-through cartridge and equilibrated with a flowing solution of 25 μM Eu, 50 μM PDA in 1% ovalbumin MOPS-buffered saline (EPOMBS) at pH 7.1. Once the fluorescent counts had stabilized, a solution of 6×10⁻⁸ M anti-E1g antibody in EPOMBS was injected across the fiber. The fluorescent signal dropped upon this addition (FIG. 6A) suggesting that antibody binding resulted in steric modification of the complex, which can displace the Eu-PDA. Injection of EPOMBS alone (dashed line) slightly increased the fluorescent signal, indicating partial displacement of bound antibody. When a solution 6×10⁻⁸ M anti-E1g antibody and 1×10⁻³ M E1g in EPOMBS was added to the same fiber, the signal rose back to the original level of Eu-PDA saturation (FIG. 6B), indicating that the free E1g has displaced all of the bound antibody and confirming that the signal response was due specifically to antibody-E1g interaction.

If a homogenous assay microwell format were employed where the complex of ligand-DTPA-Eu⁺³ is immobilized to the surface of the well and the solution contained both a Eu⁺³ and a chelate moiety, as is typically used to stabilize the immobilized complex of ligand-DTPA-Eu⁺³, UV illumination of the microwell causes fluorescence to be emitted both from the surface of the well and from the solution within the well. To limit fluorescence emission so it emanates primarily from the surface of the well, the well is precoated with a coating molecule derivatized to contain a Cy-5 molecule or other fluorescent species appropriate to allow fluorescence energy transfer (FRET) or quenching of fluorescence from the donor fluorophore, the Eu⁺³ complex (FIG. 7). An example of the coating molecule is avidin (including streptavidin, neutravidin and related species), which allows synthesis of the ligand-DTPA-Eu⁺³ complex to which biotin is appended and allow ease of construction of the immobilized species. This immobilization technique also readily lends itself to bulk synthesis of the fluorescent complex.

In use, the chelator (e.g., DTPA) can be coupled in bulk to the target analyte, such as a lead drug candidate, antigen, etc., at positions indicated as “M” in FIG. 7. As the available reactive groups on target analytes can vary, the chelator can be prepared having appropriate reactive groups, such as N-hydroxysuccinimide, hydrazide, carboxylic acid, etc. Such chemistry can occur alternatively in solid phase chemistry, which can easily allow removal of unreacted compounds. In the latter case, a readily cleavable linkage to the solid phase can be used. The liganded chelator can then be coupled to biotin and then equilibrated with Eu⁺³. The biotin-ligand-chelator-Eu⁺³ complex solution is added to the well and binds to avidin on the surface of the well. The final construct can allow fluorescence from the Eu⁺³ complex to be used to stimulate Cy5 fluorescence at the surface of the well and these binding sites can thereby be distinguished from free Eu⁺³-chelate complex remaining in solution and as such, a separate wash step is not required. This results in the disclosed method being a homogeneous assay, as no separation of bound and free must be performed. In addition, because Cy5 fluorescence emission maximum is at 670 nm and Eu⁺³'s fluorescence is at 613 nm, it is relatively straightforward to optically separate the fluorescence from the Cy5 from that of the Eu⁺³-chelate. Specifically, plates are supplied with a linker molecule derivatized with Cy5 bound to its surface. A chelate moiety containing the target recognition element is derivatized. This chelate moiety must also be designed to link to the Cy5-derivatized linker bound to the walls of the micro-wells. After incubating a short time, the compounds being screened are added to the wells, and shortly thereafter (several minutes) the Cy5 fluorescence centered at 670 nm can be measured using the plate reader. In addition, with a sufficiently fast plat reader, it should be possible to simultaneously measure the binding kinetic rate in each of the 96 wells, which can allow direct determination of association or dissociation constants.

### Example 3

This example shows the ability to use the present invention as a high-throughput screening method for compounds that act as endocrine disrupters.

Method:

Available methods for identifying substances having hormone-like activity typically are bioassays, which investigate the effect of a substance on cellular proliferation in primary cell culture, and biochemical assays, such as the effect of a substance on the ability of a natural hormone receptor to bind to ligand or to other components of a multi-molecular complex. None of these methods provides rapid, real-time determination of the hormone-like effect of a particular substance. Since hormone-like substances exert their action by binding to specific biological receptors, measurement of the binding event alone can provide a simple and rapid screen for candidate drugs. This document discloses a simple, rapid and real-time screening method, which is amenable for high-throughput screening for hormone-like substances. The method can readily be performed on 96-well, 384-well or other plates, with results scored using a fluorescence plate reader, and can be adapted to other formats, such as array chips that use fluorescence. By this method it is possible to screen large numbers of suspected endocrine disrupters, or many samples, in real time, using high-throughput methods.

Data Obtained using Fiber-optic Sensors.

To make the fluorescence measurements reported previously, a pulsed nitrogen laser and a time-resolved fluorometer were used (FIG. 1) to stimulate fluorescent chelate molecules bound to the surface of the fiber sensor (FIG. 2). Because an evanescent fiber-optic sensor was used, Eu⁺³ fluorescence was only seen if was produced by a chelate molecule attached to the sensor surface.

An E1-g/DTPAIEu complex or an ERE/DTPA/Eu complex is coupled to optical fibers. FIG. 2 shows a fiber surface with ERE attached to a surface-bound chelator.

Similar experiments were performed using estrogen receptor obtained as a crude preparation in a yeast transfection system (FIG. 8). In these experiments, 1×10⁻⁹ M ER in EPOMBS was injected into an E1g fiber cartridge, and again the signal rapidly dropped. Addition of receptor-free buffer (dashed line) resulted in restoration of the fluorescent signal.

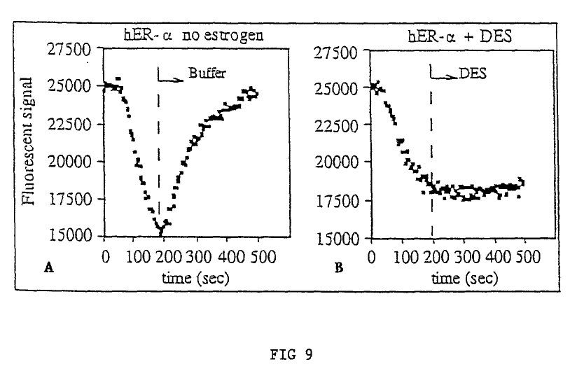

Later experiments were performed on fibers prepared such that the fluorescent complex was liganded with the ERE in the place of E1g. In these experiments, the on and off rates of the estrogen receptor for the nuclear response element were measured in the presence of a variety of receptor ligands. As an example of this data, it was found that ER-α has a significant on rate in the absence of ligand (FIG. 9, left panel), but the interaction between ER-α and the ERE is rapidly reversed by injecting buffer alone (dashed line). In the presence of a ligand such as DES (diethylstilbestrol, 1×10⁻⁸ M), the on rate is largely unchanged but the off rate becomes sharply diminished, suggesting that binding of DES stabilizes the interaction between ER-α and the ERE (FIG. 9, right panel). This result is consistent with current hypotheses regarding the molecular mechanism of action of DES and certain other ligands, and the system seems amenable for detailed study of ligand-estrogen receptor binding. These results indicate that the assay format can readily be used even with relatively large molecules like the ERE (a 32 nucleotide sequence), and promises to provide biologically relevant information that can be obtained with compounds selected from results of the screening test.

Transfer of Binding Chemistry to High Throughput Formats.

The above chemistry can readily be adapted to 20 microwell formats, such as 96- or 384-well plates. If a homogenous assay microwell format were employed where the complex of ligand-DTPA-(Eu⁺³) is immobilized to the surface of the well and the solution contained both a (Eu⁺³) and an antenna chelate moiety such as PDA, as is typically used to stabilize the immobilized complex of ligand-DTPA-(Eu⁺³), UV illumination of the microwell causes fluorescence to be emitted both from the surface of the well and from the solution within the well. To limit fluorescence emission spectra to one emanating primarily from the surface of the well, the well is precoated with a coating molecule derivatized to contain a Cy-5 molecule or other fluorescent species appropriate to allow fluorescence energy transfer (FRET) or fluorescence quenching from the donor fluorophore, (such as Eu⁺³) to occur (FIG. 7). An example of the coating molecule is avidin (including streptavidin, neutravidin and related species), which allows synthesis of the ligand-DTPA-(Eu⁺³) complex to which biotin is appended and allow ease of construction of the immobilized species. This immobilization technique also readily lends itself to bulk synthesis of the fluorescent complex.

The sensitivity of FRET to the distance, R, between donor and acceptor fluorophores, led to its use as a molecular ruler. Extensive compilations of Ro values can be found in the literature, and depending on the fluorophore pair chosen and the local environment surrounding the fluorophore, effective FRET can be observed at distances up to 100 Å. This makes FRET particularly attractive in the proposed system, as the immobilized complex can have (Eu⁺³) and Cy5 at close proximity, thereby reducing background fluorescence, as well as allowing direct measurement of a reduction in fluorescence by the acceptor fluorophore due to binding protein-mediated decoupling of fluorescence from the (Eu⁺³) mixed chelate complex.

A typical fluorescence microwell plate reader (e.g., fmax by Molecular Devices Corp.) has a detection limit for fluorescein of 2 fmol/well in 96-well plates. The dynamic range of the proposed method is limited by the surface density of the complex on microwells, about 1800 fmol/well based on a Stokes radius of about 35 Å for neutravidin, and on the transfer efficiency from Eu⁺³ to Cy5. Although Cy5 has an absorption maximum of 650 nm, it absorbs 613 nm light with 40% efficiency but does not fluoresce when illuminated with 337 nm light in the absence of FRET. The Fˆ rster equation, assuming K²⅔ (for a random distribution of orientations), predicts Rₒ=76.9 Å for this donor-acceptor pair. If, on average, one lysine per neutravidin molecule was labeled, assume a random distribution of the labels among the possible lysine residues with the average separation being midway between the closest and farthest locations, and add 10 Å for the link to Eu⁺³, the transfer efficiency can be:

$$E = \frac{{R}^{- 6}}{{R}^{- 6} + {R}_{0}^{- 6}} = 97 \%$$

Thus, the dynamic range of the assay ranges from 2 to about 1750, based upon results in microwells.

Throughout this application, various publications, including U.S. patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the described invention, the invention can be practiced otherwise than as specifically described.

### REFERENCES

U.S. Pat. No. 5,854,863 Erb J. L., Downward J. G. (1998).

U.S. Pat. No. 5,952,035 Erb J. L., Downward J. G. (1999).

Burke and Olson, “Preparation of Clone Libraries in Yeast Artificial-Chromosome Vectors” in Methods in Enzymology, Vol. 194, “Guide to Yeast Genetics and Molecular Biology”, eds. C. Guthrie and G. Fink, Academic Press, Inc., Chap. 17, pp. 251-270 (1991).

Capecchi, “Altering the genome by homologous recombination” Science 244:1288-1292 (1989).

Davies et al., “Targeted alterations in yeast artificial chromosomes for inter-species gene transfer”, Nucleic Acids Research, Vol. 20, No. 11, pp. 2693-2698 (1992).

Dickinson et al., “High frequency gene targeting using insertional vectors”, Human Molecular Genetics, Vol. 2, No. 8, pp. 1299-1302 (1993).

Duff and Lincoln, “Insertion of a pathogenic mutation into a yeast artificial chromosome containing the human APP gene and expression in ES cells”, Research Advances in Alzheimer's Disease and Related Disorders, 1995.

Huxley et al., “The human HPRT gene on a yeast artificial chromosome is functional when transferred to mouse cells by cell fusion”, Genomics, 9:742-750 (1991).

Jakobovits et al., “Germ-line transmission and expression of a human-derived yeast artificial chromosome”, Nature, Vol. 362, pp. 255-261 (1993).

Lamb et al., “Introduction and expression of the 400 kilobase precursor amyloid protein gene in transgenic mice”, Nature Genetics, Vol. 5, pp. 22-29 (1993).

Pearson and Choi, Expression of the human b-amyloid precursor protein gene from a yeast artificial chromosome in transgenic mice. Proc. Natl. Acad. Sci. USA, 1993. 90:10578-82.

Rothstein, “Targeting, disruption, replacement, and allele rescue: integrative DNA transformation in yeast” in Methods in Enzymology, Vol. 194, “Guide to Yeast Genetics and Molecular Biology”, eds. C. Guthrie and G. Fink, Academic Press, Inc., Chap. 19, pp. 281-301 (1991).

Schedl et al., “A yeast artificial chromosome covering the tyrosinase gene confers copy number-dependent expression in transgenic mice”, Nature, Vol. 362, pp. 258-261 (1993).

Strauss et al., “Germ line transmission of a yeast artificial chromosome spanning the murine a₁ (I) collagen locus”, Science, Vol. 259, pp. 1904-1907 (1993).

Gilboa, E, Eglitis, M A, Kantoff, P W, Anderson, W F: Transfer and expression of cloned genes using retroviral vectors. BioTechniques 4(6):504-512, 1986.

Cregg J M, Vedvick T S, Raschke W C: Recent Advances in the Expression of Foreign Genes in Pichia pastoris, Bio/Technology 11:905-910,1993

Culver, 1998. Site-Directed recombination for repair of mutations in the human ADA gene. (Abstract) Antisense DNA & RNA based therapeutics, February, 1998, Coronado, Calif.

Huston et al, 1991 “Protein engineering of single-chain Fv analogs and fusion proteins” in Methods in Enzymology (J J Langone, ed.; Academic Press, New York, N.Y.) 203:46-88.

Johnson and Bird, 1991 “Construction of single-chain Fvb derivatives of monoclonal antibodies and their production in Escherichia coli in Methods in Enzymology (J J Langone, ed.; Academic Press, New York, N.Y.) 203:88-99.

Mernaugh and Mernaugh, 1995 “An overview of phage-displayed recombinant antibodies” in Molecular Methods In Plant Pathology (R P Singh and U S Singh, eds.; CRC Press Inc., Boca Raton, Fla.) pp. 359-365.

### PRIOR ART

U.S. Pat. No. 4,587,223 May 6, 1986 Soini, et al. 436/536

U.S. Pat. No. 5,403,928 Apr. 4, 1995 Arrhenuis, et al. 540/128

U.S. Pat. No. 5,453,356 Sep. 26, 1995 Bard, et al. 435/6

U.S. Pat. No. 5,622,821 Apr. 22, 1997 Selvin, et al. 435/6

U.S. Pat. No. 5,637,509 Jun. 10, 1997 Hemmila, et al. 436/537

U.S. Pat. No. 5,656,433 Aug. 12, 1997 Selvin, et al. 435/6

U.S. Pat. No. 5,677,199 Oct. 14, 1997 Arrhenuis, et al. 436/536

U.S. Pat. No. 5,827,653 Oct. 27, 1998 Sammes, et al. 435/6

U.S. Pat. No. 5,830,769 Nov. 3, 1998 Wieder, et al. 435/536

U.S. Pat. No. 5,859,297 Jan. 12, 1999 Matsumoto, et al. 562/828

U.S. Pat. No. 5,998,135 Dec. 7, 1999 Rabbani, et al. 435/6

U.S. Pat. No. 5,998,146 Dec. 7. 1999 Latva, et al. 435/6

U.S. Pat. No. 6,153,384 Nov.28, 2000 Lynch, et al. 435/6

What is claimed is:

1. A method for performing a rapid, homogenous assays for monitoring the reactions of a binding target, said method comprising the steps of:
immobilizing a fluorescent-capable chelate complex that is derivatized so as to posses recognition binding ligands;
labeling the complex with a labeled second chelator that is added to the assay thereby forming a fluorescent mixed chelate;
measuring the fluorescent mixed chelate, whereby the measurement of the label enable monitoring of the reaction of the binding target.

2. The method according to claim 1, wherein said measuring step includes monitoring the binding of a first compound to a second specific recognition binding target.

3. The method according to claim 1, wherein said measuring step includes monitoring influence of a third compound on the binding of the first compound to its second recognition binding target.

4. A rapid assay for performing the method of claim 1, said assay comprising:
a first chelating molecule
a fluorescent-capable ion complexed with said first chelating molecule;
a second chelating molecule for reacting with said fluorescent-capable ion complexed with said first chelating molecule; and
measuring means for measuring fluorescent resulting from said second chelating molecule reacting with said fluorescent-capable ion complexed with said first chelating molecule.

5. The assay according to claim 4, wherein said assay is used to monitor at least a single sample.

6. The assay according to claim 4, wherein said assay is used to monitor multiple samples to identify specific compound behaviors by using it with a microwell fluorescence plate reader.

7. The assay according to claim 6, wherein said microwell fluorescence plate reader responds to fluorescence from a fluorophore excited fluorescence energy transfer from said fluorescent mixed chelate of claim 1.

8. The assay according to claim 4, wherein said first chelating molecule is a chelating molecule having multiple pendent carboxyl groups.

9. The assay according to claim 8, wherein said chelating molecule is DTPA.

10. The assay according to claim 4, wherein said fluorescent-capable ion is selected from the group consisting essentially of a europium ion, a rare earth ion, and lanthinide that is capable of forming fluorescent chelates.

11. The assay according to claim 10, wherein said europium ion forms a fluorescent chelate in conjunction with another chemical.

12. The assay according to claim 4, wherein said measuring means is selected from the group consisting essentially of a pulsed nitrogen laser and a time-resolved fluorometer.

13. The assay according to claim 4 for use in screening of libraries of compounds produced in the pharmaceutical discovery process for specific biomolecular behaviors.

14. The assay according to claim 13, wherein said biological behavior is an ability to bind selectively to specific biological moieties.

15. The assay according to claim 14, wherein said moieties are selected from the group consisting essentially of receptors, enzymes, DNA sequences, and other biological moieties.

16. A biosensor for monitoring molecular interactions between molecules, said biosensor comprising a biosensor having attached thereto a fluorescent-capable ion complexed with a first chelating molecule, whereby upon exposure to a second chelating molecule said complex becomes fluorescent.

17. The biosensor according to claim 16, wherein said molecules are estrogen receptor modulators, human estrogen receptor α (hER-α), and human estrogen receptor β (hER-β).

18. The biosensor according to claim 15, for use in monitoring the effect of a third molecular type on the interaction between molecules in a solution and molecules linked to a fluorescent-capable ion complexed with a first chelating molecule, whereby upon exposure to a second chelating molecule said complex becomes fluorescent.

19. The biosensor according to claim 16, further including fluorometer means for measuring fluorescence.

20. The biosensor according to claim 18, wherein said fluorometer uses ultra-violet light for stimulating fluoresence

21. A fluorometer for use with the biosensor according to claim 15, where said fluorometer incorporates means for:
a) Using an ultra-violet light source, such as but not limited to a laser, to stimulate fluorescence from the fluorescent complex,
b) Creating short pulses of light of said ultra-violet light source, such that the duration of each pulse so produced is very much shorter than the fluoresence lifetime of the fluorescent complex,
c) Injecting fluorescence stimulating ultra-violet light pulse into said biosensor using an optical annularizer device such as a fiber annularizer,
d) Fluorescence detection, such as but not limited to photomultipliers or photodiodes, capable of detecting the fluoresence signal of the fluorescent complex,
e) Optical filtering for substantially limiting the light signal reaching the fluoresence detection means to the fluoresence band of the fluorescent complex, and
f) Time-gated electronic measurement for processing the output of the fluoresence detection means so as to achieve a higher signal to noise ratio by electronically blocking output signals temporally close to the fluorescence stimulating light pulse and, after a suitable time delay, passing output signals from the detection means which reflect fluoresence produced by the long-lived fluorescent complex affixed to the biosensor surface.
